(19) United States
(12) Patent Application Publication
(10) Pub. No.: US 2004/0210063 A1
(43) Pub. Date: Oct. 21, 2004

(54) Novel 2,4-diaminothiazole derivatives

(76) Inventors: Andrew Neil Bowler, Gentofte (DK); Bo Falck Hansen, Virum (DK)

Correspondence Address: NOVO NORDISK PHARMACEUTICALS, INC, 100 COLLEGE ROAD WEST, PRINCETON, NJ, 08540, US

(21) Appl. No.: 10/770,705

(22) Filed: Feb. 3, 2004

(30) Foreign Application Priority Data
Aug. 3, 2001 (DK) PA 2001 01175
Jul. 22, 2002 (WO) PCT/DK02/00508

(51) Int. Cl. C07D 417/02; C07D 277/38

(52) U.S. Cl. 548/184; 544/60; 544/133; 544/369; 546/209; 548/191

(57) ABSTRACT

Novel 2,4-diaminothiazole derivatives of the general formula (I) which inhibit GSK-3 (glycogen synthase kinase-3), use of these compounds as medicaments, pharmaceutical compositions comprising the compounds and methods of treatment employing these compounds and compositions. The present compounds may be useful for the treatment of disorders, syndromes, diseases and conditions, wherein an inhibition of GSK-3 (glycogen synthase kinase-3) is beneficial, especially IGT (impaired glucose tolerance), type 1 diabetes, type 2 diabetes, obesity, Alzheimer's disease and bipolar disorder.

### CROSS REFERENCE TO RELATED APPLICATIONS

This application claims the benefit priority under 35 U.S.C. 119 of Danish application no. PA 2001 01175 filed Aug. 3, 2001 and U.S. provisional application No. 60/309,953 filed Aug. 3, 2001 and further claims priority under 35 U.S.C. 120 of international application no. PCT/DK02/00508 filed Jul. 22, 2002, the contents of all of which are fully incorporated herein by reference.

### FIELD OF THE INVENTION

The present invention relates to novel 2,4-diaminothiazole derivatives of the general formula (I) which inhibit GSK-3 (glycogen synthase kinase-3), to the use of these compounds as medicaments, to pharmaceutical compositions comprising the compounds and to methods of treatment employing these compounds and compositions. The present compounds may be useful for the treatment of disorders, syndromes, diseases and conditions, wherein an inhibition of GSK-3 is beneficial, especially IGT (impaired glucose tolerance), type 1 diabetes, type 2 diabetes, obesity, Alzheimer's disease and bipolar disorder.

### BACKGROUND OF THE INVENTION

GSK-3 is a protein-serine kinase implicated in the hormonal control of several regulatory proteins. It was first discovered by virtue of its ability to phosphorylate and inactivate glycogen synthase, the regulatory enzyme of glycogen synthesis in mammals. Since then a number of other substrates have been identified, implicating the enzyme in the regulation of several physiological processes.

GSK-3 exists in two isoforms, termed GSK-3α and GSK-3β, which are derived from distinct genes and show 85% sequence identity. Unlike many protein kinases, both GSK-3 isoforms are constitutively active in resting cells, and are primarily regulated by inactivation. Thus, it has been shown that GSK-3 is inhibited by serine phosphorylation in response to insulin and growth factors such as IGF-1 and EGF via activation of the MAP kinase cascade or via PI3 kinase dependent activation of protein kinase B.

Compounds that inhibit GSK-3 activity are useful in the treatment of diseases, disorders, syndromes and conditions, wherein such an inhibition is beneficial, eg in diseases, disorders, syndromes and conditions related to GSK-3, in diseases, disorders, syndromes and conditions related to a dysfunction of GSK-3, in diseases, disorders, syndromes and conditions in which growth factor induced inhibition of GSK-3 is insufficient, in diseases, disorders, syndromes and conditions in which glycogen synthase is insufficiently activated and in situations wherein GSK-3 inhibition could counter-regulate unwanted cellular events.

Type 1 diabetes, also known as insulin dependent diabetes mellitus (IDDM), is caused by an autoimmune destruction of insulin producing cells in the pancreas, leading to a lack of insulin. Thus, individuals with type 1 diabetes require daily injections of the hormone to sustain life. Current methods of insulin administration, however, cannot reproduce the normal p cell's ability to precisely control blood glucose and other metabolic variables. Hence, the type 1 diabetic remains susceptible to the long-term and devastating complications of diabetes, such as cardiovascular disease, retinopathy, nephropathy and neuropathy.

Type 2 diabetes, also known as non-insulin dependent diabetes mellitus (NIDDM), is the most common of all metabolic disorders and poses a major health problem worldwide. Type 2 diabetes results from defects in both insulin secretion and insulin action, but the exact underlying mechanism(s) causing the disease are not known. An elevation of hepatic glucose production contributes significantly to causing fasting hyperglycemia, whereas decreased insulin-mediated glucose uptake by muscle and fat is a major contributor to post-prandial hyperglycemia. Moreover, the metabolic fate of glucose taken up by muscle is not normal in people with type 2 diabetes. For example muscle glycogen synthase activity and glycogen synthesis have been shown to be severely impaired in type 2 diabetes. The available treatments do not allow for a complete normalisation of the metabolic state and most of them are associated with side effects. The metabolic derangements created by hyperglycemia, together with the strong association between type 2 diabetes, obesity, hypertension and hyperlipidemia, lead to an extensive list of long-term complications, including a high rate of cardiovascular death due to accelerated atherosclerosis, as well as typical complications of diabetes such as retinopathy, nephropathy and neuropathy.

Thus, there is still a considerable need for novel approaches to treat diabetes.

Recently, it has been found that GSK-3 expression is elevated in muscle of people with type 2 diabetes and that the GSK-3 expression is inversely correlated with both glycogen synthase activity and glucose disposal. Thus, an increased GSK-3 expression may contribute to the impaired glycogen synthase activity and insulin resistance that occurs in type 2 diabetes. Other recent experiments have suggested a role for GSK-3 in attenuating insulin action via its phosphorylation of insulin receptor substrate 1.

Recent studies using lithium salts also support the notion that inhibition of GSK-3 would be beneficial in the treatment of diabetes. It has been known for a long time that lithium has a stimulatory effect on glucose metabolism, most prominently on glycogen synthesis. Treatment with lithium salts has also been shown to alleviate the diabetic state in both type 1 and type 2 diabetic patients. The molecular mechanism for these effects of lithium has until recently been unknown. However, it has now been found that lithium inhibits GSK-3. Although lithium might also have effects on other molecular targets than GSK-3, this finding contributes to explain the molecular effects of lithium and supports that inhibition of GSK-3 leading to activation of glycogen synthase has significant effect on stimulation of glucose metabolism.

In conclusion, GSK-3 inhibitors may be useful for the treatment of metabolic disorders, such as IGT, type 1 diabetes and type 2 diabetes.

GSK-3 is also involved in biological pathways relating to Alzheimer's disease and GSK-3 inhibitors may be useful in the treatment thereof. Alzheimer's disease is characterized histopathologically by the presence of intraneuronal neurofibrillary tangles and the extracellular deposition of P amyloid in the brain, especially the hippocampus. The neurofibrillary tangles are made up of PHFs (paired helical filaments), the major protein subunit of which is the abnormally phosphorylated and glycosylated microtubule associated protein tau (τ). In the tangle bearing neurons in Alzheimer's disease, the normal cytoskeleton is disrupted and replaced with PHFs. GSK-3 is one of several kinases that phosphorylates tau in vitro on the abnormal sites characteristic of PHF-tau and has also been demonstrated to do this in living cells. Furthermore, the GSK-3 inhibitor lithium blocks tau hyperphosphorylation in cells. Further evidence for a role of GSK-3 in Alzheimer's disease is provided by ia (i) the association of GSK-3 with presenellin 1, (ii) reduced cytotoxicity of P amyloid protein in neuronal cells incubated with GSK-3 antisense and (iii) 50% increased expression of GSK-3 in postsynaptic supernatants of Alzheimer's disease compared to normal brain tissue.

Lithium has been used for decades in the treatment of manic depression (bipolar disorder). The mechanism of action of lithium as a mood-stabilizing agent remains unknown, although effects on biological membranes and synaptic neurotransmission have been suggested. However, GSK-3 activity could be implicated in the etiology of bipolar disorder. One mechanism by which lithium and other GSK-3 inhibitors may act to treat bipolar disorder is to increase the survival of neurons subjected to aberrantly high levels of excitation induced by the neurotransmitter glutamate. Glutamate may also be implicated in mediating neurodegeneration following acute damage, eg in cerebral ischemia, traumatic brain injury and bacterial, viral and prion infection. Excessive glutamate signalling has also been implicated in the chronic neuronal damage seen in diseases such as Huntington's chorea, Parkinson's disease, amyotrophic lateral sclerosis and multiple sclerosis. Consequently, GSK-3 inhibitors may be useful in the treatment of these and other neurodegenerative disorders. In connection with this it should be noted that lithium has a variety of biological effects that, if mediated through the inhibition of GSK-3, could provide an even broader application of GSK-3 inhibitors.

Furthermore, GSK-3 has been shown to phosphorylate the transcription factor NF-AT, which participates in the activation of early immune response genes. Phosphorylation prevents translocation of NF-AT to the nucleus and thus blocks early immune responses. Thus, GSK-3 inhibitors may prolong and potentiate the immunostimulatory effect of certain cytokines and such an effect could be beneficial in the use of cytokines for cancer or immunotherapy.

Different classes of compounds have been disclosed as inhibitors of GSK-3, cf ia WO 98/16528, WO 99/65897, WO 00/21927, WO 01//09106, WO 00/38675, WO 01/44206 and WO 01/44246. These compounds differ structurally from the present compounds.

WO 99/21845 discloses 4-aminothiazole derivatives and their use as inhibitors of cyclin-dependent kinases (CDKs). The compounds are stated to be effective for the treatment of ia cancer. The compounds differ structurally from the present compounds. However, Table I discloses one compound (I(6)), of the following structure:

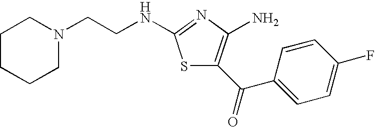

No CDK inhibition was observed for the compound at the tested concentration.

WO 00/75120 discloses diaminothiazoles and their use for inhibiting protein kinases. The compounds are stated to be useful for the treatment of disease conditions associated with tumor growth, cell proliferation or angiogenesis, such as cancer. The compounds differ structurally from the present compounds.

In view of the art's interest in GSK-3 inhibitors and the great potential thereof, the identification of potent and specific GSK-3 inhibitors would be a highly desirable contribution to the art. The present invention provides such a contribution to the art being based on the finding that the 2,4-diaminothiazole derivatives of the general formula (I) potently and specifically inhibit GSK-3.

The present compounds are accordingly useful in the treatment of a wide range of disorders, syndromes, diseases and conditions in which an inhibition of GSK-3 is beneficial.

Definitions

The following is a detailed definition of the terms used to describe the compounds of the invention.

“Halogen” designates an atom selected from the group consisting of F, Cl, Br and 1.

The term “C₁₋₆-alkyl” in the present context designates a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₁₋₆-alkoxy” in the present context designates a group —O—C₁₋₆-alkyl, wherein C₁₋₆-alkyl is as defined above. Representative examples include, but are not limited to, methoxy, ethoxy, n-propoxy, isopropoxy, butoxy, isobutoxy, sec-butoxy, tert-butoxy, n-pentoxy, isopentoxy, neopentoxy, tert-pentoxy, n-hexoxy, isohexoxy and the like.

The term “C₂₋₆-alkenyl” as used herein represents a branched or straight hydrocarbon group having from 2 to 6 carbon atoms and at least one double bond. Examples of such groups include, but are not limited to, vinyl, 1-propenyl, 2-propenyl, isopropenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

The term “C₂₋₆-alkynyl” as used herein represents a branched or straight hydrocarbon group having from 2 to 6 carbon atoms and at least one triple bond. Examples of such groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 5-hexynyl, 2,4-hexadiynyl and the like.

The term “C₃₋₈-cycloalkyl” as used herein represents a saturated carbocyclic group having from 3 to 8 carbon atoms. Representative examples are cyclopropyl, cyclobutyl, cyclopentyl, cyclohexyl, cycloheptyl, cyclooctyl and the like.

The term “C₃₋₈-heterocyclyl” as used herein represents a saturated 3 to 8 membered ring containing one or more heteroatoms selected from nitrogen, oxygen and sulphur. Representative examples are pyrrolidinyl, piperidyl, piperazinyl, morpholinyl, thiomorpholinyl, aziridinyl, tetrahydrofuranyl and the like.

The term “aryl” as used herein represents a carbocyclic aromatic ring system such as phenyl, biphenyl, naphthyl, anthracenyl, phenanthrenyl, fluorenyl, indenyl, pentalenyl, azulenyl, biphenylenyl and the like. Aryl is also intended to include the partially hydrogenated derivatives of the carbocyclic aromatic systems enumerated above. Non-limiting examples of such partially hydrogenated derivatives are 1,2,3,4-tetrahydronaphthyl, 1,4-dihydronaphthyl and the like.

The term “heteroaryl” as used herein is intended to include heterocyclic aromatic ring systems containing one or more heteroatoms selected from nitrogen, oxygen and sulphur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyi, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

“Aryl-C₁₋₆-alkyl”, “heteroaryl-C₁₋₆-alkyl” etc. means C₁₋₆-alkyl as defined above, substituted by an aryl or heteroaryl as defined above, for example:

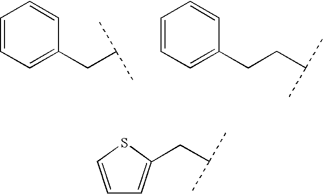

Certain of the above defined terms may occur more than once in the structural formulae, and upon such occurrence each term shall be defined independently of the other.

The term “GSK-3” as used herein is intended to mean GSK-3α and/or GSK-β.

The term “treatment” as used herein means the management and care of a patient for the purpose of combating a disease, disorder, syndrome or condition. The term is intended to include the delaying of the progression of the disease, disorder, syndrome or condition, the alleviation or relief of symptoms and complications, and/or the cure or elimination of the disease, disorder, syndrome or condition. The patient to be treated is preferably a mammal, in particular a human being.

### DESCRIPTION OF THE INVENTION

The present invention relates to a compound of the general formula (I):

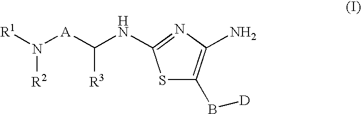

wherein

A is a valence bond or C₁₋₆-alkylene,

(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from

hydrogen,

oxo,

C₁₋₆-alkyl, C₂₋₆-alkenyl, C₂₋₆-alkynyl,

which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆-alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,

wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆-alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,

wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,

and R³ is hydrogen,

(ii) or R¹ is hydrogen, —C(═O)OR¹⁰, —C(═O)R¹⁰, C₁₋₆-alkyl, aryl-C₃₋₈-alkyl, C₃₋₈-cycloalkyl-C₁₋₆-alkyl, heteroaryl-C₁₋₆-alkyl or C₃₋₈-heterocyclyl-C₁₋₆-alkyl,

wherein R¹⁰ is C₁₋₆-alkyl, C₂₋₆-alkenyl or C₂₋₆-alkynyl, which may optionally be substituted with one or two substituents independently selected from

hydroxy, halogen, cyano, nitro, —NR¹¹R¹², —C(═O)NR¹¹R¹², —OC(═O)NR¹¹R¹²—OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,

wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,

and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from

hydrogen,

oxo,

C₁₋₆-alkyl, C₂₋₆-alkenyl, C₂₋₆-alkynyl,

which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, Cl-alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,

wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

wherein the ring moieties may optionally be substituted with one to three substituents independently selected from

hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,

wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,

(iii) or R¹ and R² which may be the same or different independently are selected from hydrogen, —C(═O)OR¹⁹—C(═O)R¹⁹ and C₁₋₆-alkyl,

wherein R¹⁹ is C₁₋₆-alkyl, C₂₋₆-alkenyl or C₂₋₆-alkynyl, which may optionally be substituted with one or two substituents independently selected from

hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,

wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,

and R³ is hydrogen,

B is a valence bond, —C(═O)—, —S(═O)— or —S(═O)₂—,

D is

hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,

wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR²⁴R²⁵, —C(═O)NR²⁴R²⁵, —OC(═O)NR²⁴R²⁵—OCH₂C(═O)NR²⁴R²⁵, C₁₋₆alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,

wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-Cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,

wherein the ring moieties may optionally be substituted with one to three substituents selected from

hydroxy, halogen, cyano, nitro, —NR²⁶R²⁷, —C(═O)NR²⁶R²⁷, —OC(═O)NR²⁶R²⁷—OCH₂C(═O)NR²⁶R²⁷, C₁₋₆alkoxy, —C(═O)OR²⁶, —C(═O)R²⁶, —NHC(═O)R²⁶, —CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,

wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆-alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,

wherein R²⁸ and R²⁹ which may be the same or different independently are selected from hydrogen and C₁₋₆-alkyl, or R²³ and R²⁹, together with the nitrogen atom to which they are attached, form a 3 to 8 membered cyclic ring optionally containing one or two further heteroatoms selected from oxygen, sulphur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆alkoxy, C₃₋₈-Cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆-alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,

wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,

with the proviso that the compound must not be

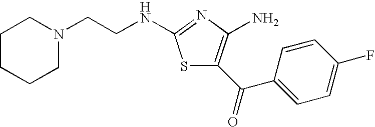

as well as any optical or geometric isomer or tautomeric form thereof including mixtures of these or a pharmaceutically acceptable salt thereof.

In one embodiment of the invention R² and R³ are both hydrogen, and R¹ is —C(═O)OR¹⁹, wherein R¹⁹ is as defined for formula (I). In an embodiment thereof R¹⁹ is C₁₋₆-alkyl.

In another embodiment of the invention R³ is hydrogen, and R¹ and R², together with the nitrogen atom to which they are attached, form a ring

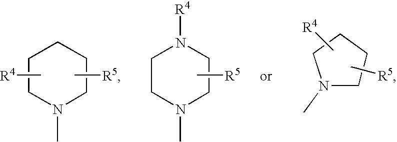

wherein R⁴ and R⁵ are as defined for formula (I). In an embodiment thereof R⁴ and R⁵ are independently selected from hydrogen, C₁₋₆-alkyl, phenyl-C₁₋₆-alkyl and oxo. In a further embodiment thereof R⁴ is hydrogen or C₁₋₆-alkyl, and R⁵ is hydrogen or oxo.

In yet another embodiment of the invention R³ is hydrogen, and R¹ and R², together with the nitrogen atom to which they are attached, form a ring

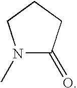

In still another embodiment of the invention R² and R³, together with A and the nitrogen atom and the carbon atom, respectively, to which they are attached, form a ring

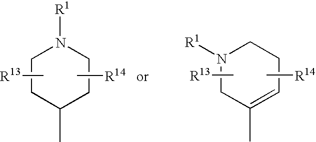

wherein R¹, R¹³ and R¹⁴ are as defined for formula (I).

In an embodiment thereof R² and R³, together with A and the nitrogen atom and the carbon atom, respectively, to which they are attached, form a ring

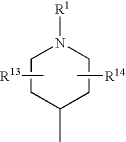

wherein R¹, R¹³ and R¹⁴ are as defined for formula (I).

In an embodiment thereof R¹ is hydrogen, C₁₋₆-alkyl, phenyl-C₁₋₆-alkyl or —C(═O)OR¹⁰, wherein R¹⁰ is as defined in claim 1, and R¹³ and R¹⁴ are independently hydrogen, C₁₋₆-alkyl, phenyl-C₁₋₆-alkyl or oxo. In a further embodiment thereof R¹ is hydrogen or —C(═O)O—C₁₋₆-alkyl, and R¹³ and R¹⁴ are hydrogen.

In a further embodiment of the invention R¹, R² and R³ are hydrogen.

In still a further embodiment of the invention A is C₁₋₆-alkylene. In an embodiment thereof A is methylene or ethylene. In yet a further embodiment thereof A is ethylene.

In yet a further embodiment of the invention B is —C(═O)—.

In still a further embodiment of the invention D is C₃₋₈-cycloalkyl, heteroaryl or aryl, which may optionally be substituted as defined for formula (I).

In an embodiment thereof D is C₃₋₈-cycloalkyl, heteroaryl or aryl, which may optionally be substituted as defined for formula (I), but not in the positions adjacent to the point of attachment of D to B.

In another embodiment thereof D is cyclopropyl, thienyl or phenyl, which may optionally be substituted as defined for formula (I).

In still another embodiment thereof D is cyclopropyl.

In yet another embodiment thereof D is thienyl, which is substituted with halogen.

In yet a further embodiment thereof D is phenyl, which is optionally substituted with

hydroxy, halogen,

heteroaryl-C₁₋₆alkoxy, aryl-C₁₋₆-alkoxy, wherein the ring moieties are optionally substituted as defined for formula (I). In one embodiment thereof D is phenyl which is optionally substituted with halogen or benzyloxy, wherein the ring moiety of benzyloxy is optionally substituted as defined for formula (I). In a further embodiment thereof D is phenyl, which is substituted with benzyloxy.

The compounds of the present invention may have one or more asymmetric centres and it is intended that any optical isomers, as separated, pure or partially purified optical isomers or racemic mixtures thereof are included within the scope of the invention.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms, which the compounds are able to form, are included within the scope of the present invention.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulphuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulphonic, ethanesulphonic, tartaric, ascorbic, pamoic, bismethylene salicylic, ethanedisulphonic, gluconic, citraconic, aspartic, stearic, palmitic, EDTA, glycolic, p-aminobenzoic, glutamic, benzenesulphonic, p-toluenesulphonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates, which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the formula (I). Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The present compounds are useful for the treatment of hyperglycemia; IGT; Syndrome X; type 1 diabetes; type 2 diabetes; conditions with dyslipidemia including diabetic dyslipidemia; and obesity. Furthermore, they may be useful for the treatment of albuminuria; polycystic ovary syndrome, cardiovascular diseases such as cardiac hypertrophy, hypertension and arteriosclerosis including atherosclerosis; gastrointestinal disorders; acute pancreatitis; and appetite regulation or energy expenditure disorders.

They may also find use in the treatment of bipolar disorder (manic depressive syndrome), mania, Alzheimer's disease, bipolar disorder, Huntington's chorea, Parkinson's disease, amyotrophic lateral sclerosis, multiple sclerosis, leukopenia, anxiety, movement disorder, aggression, psychosis, seizures, panic attacks, hysteria or sleep disorders. Furthermore, they may be useful as contraceptives, cf WO 97/41854, and for the treatment of cancer, hair-loss and neurotraumatic diseases, such as acute stroke, cf WO 00/21927. Thus, in another aspect the invention relates to the use of a compound of the general formula (I′):

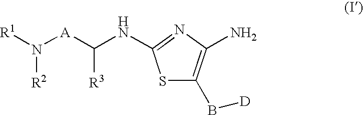

wherein

A is a valence bond or C₁₋₆-alkylene,

(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from

hydrogen,

oxo,

C₁₋₆-alkyl, C₂₋₆-alkenyl, C₂₋₆-alkynyl,

which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆-alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,

wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

wherein the ring moieties may optionally be substituted with one to three substituents independently selected from hydroxy, halogen, cyano, nitro, —NR⁸R⁹, —C(═O)NR⁸R⁹, —OC(═O)NR R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,

wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,

and R³ is hydrogen,

(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₄-heterocyclyl-C₁₋₆-alkyl,

wherein R¹⁰ is C₁₋₆-alkyl, C₂₋₆-alkenyl or C₂₋₆-alkynyl, which may optionally be substituted with one or two substituents independently selected from

hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,

wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,

and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from

hydrogen,

oxo,

C₁₋₆-alkyl, C₂₋₆-alkenyl, C₂₋₆-alkynyl,

which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,

wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-hetercyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

wherein the ring moieties may optionally be substituted with one to three substituents independently selected from

hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,

wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,

(iii) or R¹ and R² which may be the same or different independently are selected from hydrogen, —C(═O)OR¹⁹—C(═O)R¹⁹ and C₁₋₆-alkyl,

wherein R¹⁹ is C₁₋₆-alkyl, C₂₋₆-alkenyl or C₂₋₆-alkynyl, which may optionally be substituted with one or two substituents independently selected from

hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,

wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,

and R³ is hydrogen,

B is a valence bond, —C(═O)—, —S(═O)— or —S(═O)₂—,

D is

hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,

wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR²⁴R²⁵, —C(═O)NR²⁴R²⁵, —OC(═O)NR²⁴R²⁵—OCH₂C(═O)NR²⁴R²⁵, C₁₋₆alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,

wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-Cl-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,

wherein the ring moieties may optionally be substituted with one to three substituents selected from

hydroxy, halogen, cyano, nitro, —NR²⁶R²⁷, —C(═O)NR²⁶R²⁷, —OC(═O)NR²⁶R²⁷—OCH₂C(═O)NR²⁶R²⁷, C₁₋₆-alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,

wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,

wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₃-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

wherein the ring moieties may optionally be substituted with one to three substituents selected from hydroxy, halogen, cyano, nitro, —NR³⁰R³¹, —C(═O)NR³⁰R³¹, —OC(═O)NR³⁰R³, —OCH₂C(═O)NR³⁰R³, C₁₋₆-alkyl, C₂₋₆-alkenyl, C₂₋₆-alkynyl, C₁₋₆-alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³³, —S(═O)₂NH₂,

wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,

as well as any optical or geometric isomer or tautomeric form thereof including mixtures of these or a pharmaceutically acceptable salt thereof for the preparation of a pharmaceutical composition for the treatment of diseases, disorders, syndromes and conditions, wherein an inhibition of GSK-3 is beneficial.

In a further preferred embodiment of the invention the compounds of the general formula (I′) are used for the manufacture of a pharmaceutical composition for the treatment of diseases, disorders, syndromes and conditions related to GSK-3.

In a further preferred embodiment of the invention the present compounds of the general formula (I′) are used for the manufacture of a pharmaceutical composition for the treatment of diseases, disorders, syndromes and conditions, wherein growth factor induced inhibition of GSK-3 is insufficient.

In another preferred embodiment of the invention the present compounds of the general formula (I′) are used for the preparation of a pharmaceutical composition for the treatment of diseases, disorders, syndromes and conditions, wherein glycogen metabolism exhibits abnormalities.

In another preferred embodiment of the invention the present compounds of the general formula (I′) are used for the preparation of a pharmaceutical composition for the treatment of diseases, disorders, syndromes and conditions, wherein glycogen synthase is insufficiently activated.

In a further preferred embodiment of the invention the present compounds of the general formula (I′) are used for the preparation of a pharmaceutical composition for the treatment of diseases, disorders, syndromes and conditions involving elevated blood glucose. The compounds are effective in lowering both fasting and postprandial blood glucose.

In still a further preferred embodiment of the invention the present compounds of the general formula (I′) are used for the preparation of a pharmaceutical composition for the treatment of hyperglycemia.

In yet a further preferred embodiment of the invention the present compounds of the general formula (I′) are used for the preparation of a pharmaceutical composition for the treatment of IGT.

In another preferred aspect the invention the present compounds of the general formula (I′) are used for the preparation of a pharmaceutical composition for the treatment of type 2 diabetes. Such treatment includes ia the delaying of the progression from IGT to type 2 diabetes as well as the delaying of the progression from non-insulin requiring type 2 diabetes to insulin requiring type 2 diabetes.

In a further preferred aspect of the invention the present compounds of the general formula (I′) are used for the preparation of a pharmaceutical composition for the treatment of type 1 diabetes. Such treatment is normally accompanied by insulin therapy.

Furthermore, the present compounds of the general formula (I′) may be used for the preparation of a pharmaceutical composition for the treatment of obesity.

In another aspect of the invention the present compounds of the general formula (I′) may be used for the preparation of a pharmaceutical composition for the treatment of Alzheimer's disease.

In another aspect of the invention the present compounds of the general formula (I′) may be used for the preparation of a pharmaceutical composition for the treatment of bipolar disorder.

In a further aspect of the invention the present compounds are administered in combination with diet and/or exercise.

In yet a further aspect of the invention the present compounds are administered in combination with one or more further pharmacologically active substances in any suitable ratios. Such further active agents may be selected from antidiabetic agents, antihyperlipidemic agents, antiobesity agents, antihypertensive agents and agents for the treatment of complications resulting from or associated with diabetes. Furthermore, they may be administered in combination with one or more further pharmacologically active substances selected from agents for the treatment of Alzheimer's disease and agents for the treatment of bipolar disorder. Such combined administration may be in separate preparations or in a single preparation, as appropriate.

Suitable antidiabetics comprise insulin, GLP-1 (glucagon like peptide-1) derivatives such as those disclosed in WO 98/08871 (Novo Nordisk A/S), which is incorporated herein by reference as well as orally active hypoglycaemic agents.

The orally active hypoglycaemic agents preferably comprise imidazolines, sulphonylureas, biguanides, meglitinides, oxadiazolidinediones, thiazolidinediones, insulin sensitizers, α-glucosidase inhibitors, agents acting on the ATP-dependent potassium channel of the β-cells eg potassium channel openers such as those disclosed in WO 97/26265, WO 99/03861 and WO 00/37474 (Novo Nordisk A/S), which are incorporated herein by reference, or mitiglinide, or a potassium channel blocker, such as BTS-67582, nateglinide, glucagon antagonists such as those disclosed in WO 99/01423 and WO 00/39088 (Novo Nordisk A/S and Agouron Pharmaceuticals, Inc.), which are incorporated herein by reference, GLP-1 agonists such as those disclosed in WO 00/42026 (Novo Nordisk A/S and Agouron Pharmaceuticals, Inc.), which is incorporated herein by reference, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, compounds modifying the lipid metabolism such as antilipidemic agents, compounds lowering food intake, PPAR (peroxisome proliferator-activated receptor) and RXR (retinoid X receptor) agonists, such as ALRT-268, LG-1268 or LG-1069.

In one embodiment of the invention the present compounds are administered in combination with insulin.

In a further embodiment of the invention the present compounds are administered in combination with a sulphonylurea eg tolbutamide, chlorpropamide, tolazamide, glibenclamide, glipizide, glimepiride, glicazide or glyburide.

In another embodiment of the invention the present compounds are administered in combination with a biguanide eg mefformin.

In yet another embodiment of the invention the present compounds are administered in combination with a meglitinide eg repaglinide or nateglinide.

In still another embodiment of the invention the present compounds are administered in combination with a thiazolidinedione insulin sensitizer eg troglitazone, ciglitazone, pioglitazone, rosiglitazone, isaglitazone, darglitazone, englitazone, CS-011/CI-1037 or T 174 or the compounds disclosed in WO 97/41097, WO 97/41119, WO 97/41120, WO 00/41121 and WO 98/45292 (Dr. Reddy's Research Foundation), which are incorporated herein by reference.

In still another embodiment of the invention the present compounds are administered in combination with an insulin sensitizer eg such as GI 262570, YM440, MCC-555, JTT-501, AR-H039242, KRP-297, GW409544, CRE-16336, AR-H049020, LY510929, MBX-102, CLX-0940, GW-501516 or the compounds disclosed in WO 99/19313, WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In a further embodiment of the invention the present compounds are administered in combination with an α-glucosidase inhibitor eg voglibose, emiglitate, miglitol or acarbose.

In another embodiment of the invention the present compounds are administered in combination with an agent acting on the ATP-dependent potassium channel of the β-cells eg tolbutamide, glibenclamide, glipizide, glicazide, BTS-67582 or repaglinide.

In still another embodiment of the invention the present compounds are administered in combination with an antilipidemic agent eg cholestyramine, colestipol, clofibrate, gemfibrozil, lovastatin, pravastatin, simvastatin, probucol or dextrothyroxine.

In another aspect of the invention, the present compounds are administered in combination with more than one of the above-mentioned compounds eg in combination with metformin and a sulphonylurea such as glyburide; a sulphonylurea and acarbose; nateglinide and mefformin; acarbose and metformin; a sulphonylurea, mefformin and troglitazone; insulin and a sulphonylurea; insulin and mefformin; insulin, mefformin and a sulphonylurea; insulin and troglitazone; insulin and lovastatin; etc.

Thus, in a further aspect of the invention the present compounds are administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, MC3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline re-uptake inhibitors, mixed serotonin and noradrenergic compounds, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, RXR (retinoid X receptor) modulators, TR β agonists, AGRP (Agouti related protein) inhibitors, H3 histamine antagonists such as those disclosed in WO 00/42023, WO 00/63208 and WO 00/64884 (Novo Nordisk A/S and Boehringer Ingelheim International GmbH), which are incorporated herein by reference, opioid antagonists (such as naltrexone), exendin-4, GLP-1 and ciliary neurotrophic factor.

In one embodiment of the invention the antiobesity agent is leptin.

In another embodiment the antiobesity agent is dexamphetamine or amphetamine.

In another embodiment the antiobesity agent is fenfluramine or dexfenfluramine.

In still another embodiment the antiobesity agent is sibutramine.

In a further embodiment the antiobesity agent is orlistat.

In another embodiment the antiobesity agent is mazindol or phentermine.

In still another embodiment the antiobesity agent is phendimetrazine, diethylpropion, fluoxetine, bupropion, topiramate or ecopipam.

Furthermore, in another aspect of the invention the present compounds are administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

In another aspect of the invention the present compounds are administered in combination with one or more agents for the treatment of Alzheimer's disease. Examples of such agents are tacrine, donepezil, haloperidol, olanzapine, quetiapine, risperidone, alprazolam, buspirone, diazepam, lorazepam, amitriptyline, bupropion, desipramine, fluoxetine, fluvoxamine, nefazodone, nortriptyline, paroxetine, sertraline and trazodone.

In yet another aspect of the invention the present compounds are administered in combination with one or more agents for the treatment of bipolar disorder. Examples of such agents are lithium, valproate, divalproex, carbamazepine, antipsychotic drugs such as haloperidol and perphenazine, antianxiety agents such as lorazepam and clonazepam, antidepressants such as bupropion, fluoxetine, fluvoxamine, paroxetine, sertraline, mirtazepine, pheneizine, tranylcypromine, nefazodone, amitriptyline, desipramine, imipramine, nortriptyline and venlafaxine.

It should be understood that any suitable combination of the compounds according to the invention with diet and/or exercise, one or more of the above-mentioned compounds and optionally one or more other pharmacologically active substances are considered to be within the scope of the present invention.

Pharmaceutical Compositions

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain of from 0.05 to about 1000 mg, preferably from about 0.1 to about 500 mg, and more preferred from about 0.5 mg to about 200 mg.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the formula (I) contains a free base such salts are prepared in a conventional manner by treating a solution or suspension of a free base of the formula (I) with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the novel compounds of the formula (I) in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the novel compounds of the formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet, which may be prepared by conventional tabletting techniques, may contain:

| Core: | | |
|---|---|---|
| Active compound (as free compound or salt thereof) | | 5.0 mg |
| Lactosum Ph. Eur. | | 67.8 mg |
| Cellulose, microcryst. (Avicel) | | 31.4 mg |
| Amberlite ® IRP88* | | 1.0 mg |
| Magnesii stearas Ph. Eur. | | q.s. |
| Coating: | | |
| Hydroxypropyl methylcellulose | approx. | ’9 mg |
| Mywacett 9-40 T** | approx. | 0.9 mg |

* Polacrillin potassium NF, tablet disintegrant, Rohm and Haas.

** Acylated monoglyceride used as plasticizer for film coating.

If desired, the pharmaceutical composition of the invention may comprise the compound of the formula (I) in combination with further pharmacologically active substances such as those described in the foregoing.

The present invention is further illustrated by the following representative examples which are, however, not intended to limit the scope of the invention in any way.

### EXAMPLES

The compounds used as starting materials are either known compounds or compounds, which can be prepared by methods known per se. NMR spectra were recorded on a Bruker 300 MHz instrument. Flash chromatography was carried out on Merck silica gel 60 (Art 9385).

HPLC-MS Method A:

Instruments:

Sciex API 100 single quadropole mass spectrometer,

Applied Biosystems 785A UV detector,

Sedex 55 evaporative light scattering detector.

Column: YMC ODS-A 120 Å s—5μ (50 mm×3 mm id). Gradient: 5%-90% acetonitrile (with 0.05% TFA) during 7.5 min. UV detection at 214 nm.

HPLC-MS Method B:

Column: Waters Xterra MS C-18×3 mm id. Linear gradient 10%-100% in 7.5 min, acetonitrile, 0.01% TFA, flow rate 1.0 ml/min. Detection 210 nm (analog output from diode array detector), MS-detection ionisation mode API-ES, scan 100-1000 amu step 0.1 amu.

In the examples and assays the following terms are intended to have the following meanings:

Boc: tert-butyloxycarbonyl

DMF: N,N-dimethylformamide

DMSO: dimethyl sulphoxide

EtOAc: ethyl acetate

HCl: hydrogen chloride

M.p.: melting point

TFA: trifluoroacetic acid

General Procedure (A)

The preparation of the compounds of the formula (Ia) according to this invention wherein B is —C(═O)— may be illustrated in the scheme below and may be achieved using the method described by Gewald K. et al (J. Prakt. Chem., 35, 1967, pp 97-104). An isothiocyanate of formula (III) is reacted together with a bromomethylketone of formula (V) to give products of the general formula (Ia) (step C). When not commercially available, the starting materials of formulae (III) and (V) are prepared using literature procedures, as cited under the individual examples and as illustrated in steps A and B below.

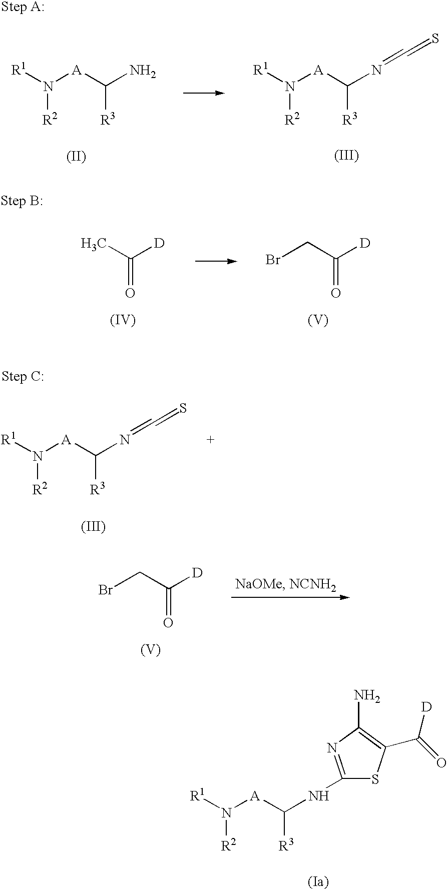

When R¹ and R² are both hydrogen the amino group formed should be protected with a suitable protecting group, such as Boc, before carrying out step A. After step C the protecting group can be removed in a conventional manner to form the compounds of the formula (Ia) wherein R¹ and R² are both hydrogen.

### Example 1

[3-(4-Amino-5-cyclopropanecarbonylthiazol-2-ylamino)propyl]carbamic acid tert-butyl ester

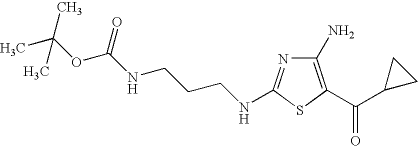

The method described by Gewald K. et al (J. Prakt. Chem., 35, 1967, pp 97-104) was employed using 2-bromo-1-cyclopropylethanone and N-Boc-isothiocyanatopropylamine. 2-Bromo-1-cyclopropylethanone was itself prepared using a slight modification to the literature procedure described by Calverley M. J. (Tetrahedron, 43, 20, 1987, 4609-4619). A temperature of 10-15° C. was used throughout the addition of bromine.

The title compound was obtained in 32% yield after chromatography using ethyl acetate/heptane (2:1) as eluant.

M.p. 144-146° C.; ¹H NMR (300 MHz; DMSO-d₆): δ 0.75 (2H, m, CH₂), 0.82 (2H, m, CH₂), 1.39 (9H, s, ^{t-}Bu), 1.68 (3H, m, CH₂ and CH), 2.98 (2H, dd, CH₂), 3.23 (2H, dd, CH₂), 6.87 (1H, br t, NH), 7.61 (2H, br s, NH₂), 8.48 (1H, t, NH).

The following compounds, unless specified otherwise, were prepared as described in Example 1 using the appropriate starting materials.

### Example 2

[2-(4-Amino-5-cyclopropanecarbonylthiazol-2-ylamino)ethyl]carbamic acid tert-butyl ester

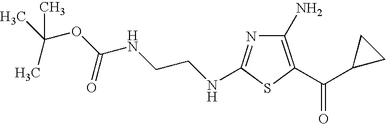

¹H NMR (300 MHz; DMSO-d₆): ¹H NMR (300 MHz; DMSO-d₆): δ 0.75 (2H, m, CH₂), 0.82 (2H, m, CH₂), 1.40 (9H, s, ^{t-}Bu), 1.64 (1H, m, CH), 3.12 (2H, dd, CH₂), 3.28 (2H, br, CH₂), 6.94 (1H, br t, NH), 7.62 (2H, br s, NH₂), 8.49 (1H, t, NH).

### Example 3

3-[4-Amino-5-(5-chlorothiophene-2-carbonyl)thiazol-2-ylamino]propylcarbamic acid tert-butyl ester

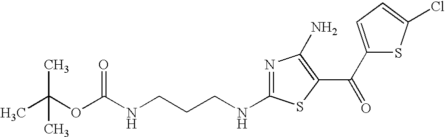

¹H NMR (300 MHz; DMSO-d₆): δ 1.39 (9H, s, ^{t-}Bu), 1.68 (2H, ddd, CH₂), 2.98 (2H, dd, CH₂), 3.30 (2H, br, CH₂), 6.87 (1H, brt, NH), 7.18 (1H, d, Ar—H), 7.32 (1H, brd, Ar—H), 7.85-8.55 (2H, br s, NH₂), 8.88 (1H, t, NH); HPLC-MS (ESI): m/z 418 [M+H]⁺; Rₜ=5.73 min (Method A).

### Example 4

4-(4-Amino-5-cyclopropanecarbonylthiazol-2-ylamino)piperidine-1-carboxylic acid tert-butyl ester

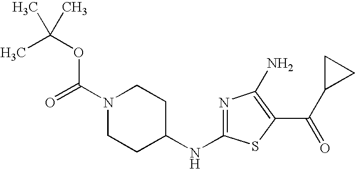

The starting material, 4-isothiocyanatopiperidine-1-carboxylic acid tert-butyl ester, was first prepared, from the corresponding amine using di-2-pyridylthionocarbonate, according to the method of Kim, S. and Yi, K. Y. (Tetrahedron Lett., 26 (13), 1985, pp. 1661-1664). The title compound was obtained after chromatography using ethyl acetate/heptane (1:1) as eluant.

¹H NMR (300 MHz; DMSO-d₆): δ 0.75 (2H, m, CH₂), 0.82 (2H, m, CH₂), 1.34 (2H, m, CH₂), 1.39 (9H, s, ^{t-}Bu), 1.62 (1H, m, CH), 1.81 (2H, dd, CH₂), 2.88 (2H, m, CH₂), 3.73 (1H, br, CH), 3.88 (2H, dd, CH₂), 7.64 (2H, br s, NH₂), 8.52 (1H, d, NH); HPLC-MS (ESI): m/z 367 [M+H]⁺; Rₜ=4.95 min (Method A).

### Example 5

4-[4-Amino-5-(3-benzyloxybenzoyl)thiazol-2-ylamino]piperidine-1-carboxylic acid tert-butyl ester

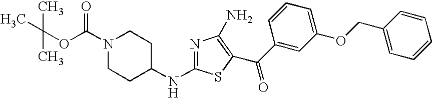

The starting material, 1-(3-benzyloxyphenyl)-2-bromoethanone, was first prepared, from the corresponding ketone using copper (II) bromide, according to the method of King, L. C. et al (J. Org. Chem., 1964, 29, pp 3459-3461). The title compound was obtained after chromatography using ethyl acetate/heptane (1:1) as eluant.

¹H NMR (300 MHz; DMSO-d₆): δ 1.34 (2H, m, CH₂), 1.39 (9H, s, ^{t-}Bu), 1.88 (2H, m, CH₂), 2.88 (2H, m, CH₂), 3.73 (1H, br, CH), 3.87 (2H, dd, CH₂), 5.65 (2H, s, OCH₂), 7.07-7.48 (9H, m, Ar—H), 7.70-8.50 (2H, br, NH₂), 8.62 (1H, br d, NH); HPLC-MS (ESI): m/z 509 [M+H]⁺; Rₜ=7.08 min (Method A).

### Example 6

{3-[4-Amino-5-(3-benzyloxybenzoyl)thiazol-2-ylamino]propyl}carbamic acid tert-butyl ester

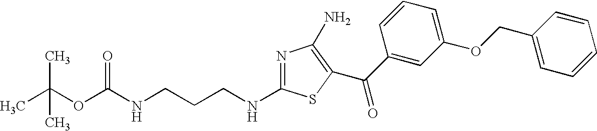

An intermediate, S-(3-benzyloxybenzoyl)methyl-N′-cyano-N″-propylcarbamic acid tert-butyl ester isothiourea was isolated when the aforementioned method of Gewald K. et al was employed.

¹H NMR (300 MHz; DMSO-d₆): δ 1.33 (9H, s, ^{t-}Bu), 1.55 (2H, m, CH₂), 2.75 (2H, dd, CH₂), 2.90 (1H, m, CH), 3.19 (1H, m, CH), 3.70 (2H, AB, dd, SCH₂), 5.63 (2H, s, OCH₂), 6.71 (1H, t, NH), 7.05-7.48 (9H, m, Ar—H), 7.63 (1H, s, NH).

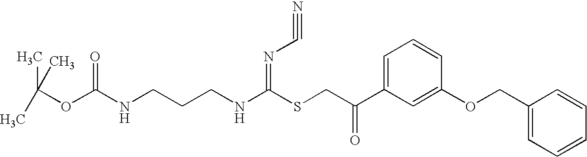

The crude intermediate was refluxed overnight in ethylacetate with 2 equivalents of triethylamine. The title compound was obtained on cooling and addition of water.

¹H NMR (300 MHz; DMSO-d₆): δ 1.41 (9H, s, ^{t-}Bu), 1.70 (2H, ddd, CH₂), 3.03 (2H, dd, CH₂), 3.28 (2H, br, CH₂), 5.20 (2H, s, OCH₂), 6.85 (1H, br t, NH), 7.08-7.48 (9H, m, Ar—H), 7.81-8.4 (2H, br s, NH₂), 8.60 (1H, br t, NH); HPLC-MS (ESI): m/z 483 [M+H]⁺; Rₜ=4.23 min (Method B).

### Example 7

1-{3-[4-Amino-5-(5-chlorothiophene-2-carbonyl)thiazol-2-ylamino]propyl}pyrrolidin-2-one

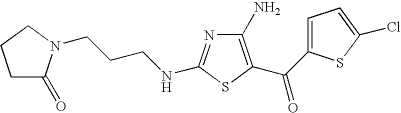

The starting material, 1-(3-isothiocyanatopropyl)pyrrolidin-2-one, was first prepared, from the corresponding amine, using di-2-pyridylthionocarbonate, according to the method of Kim, S. and Yi, K. Y. (Tetrahedron Lett., 26 (13), 1985, pp. 1661-1664). The product was found to precipitate from the reaction mixture. This was filtered off and stirred in hot ethanol before warm filtration offered the title compound in 40% yield.

M.p. 224-225° C.; HPLC-MS (ESI): m/z 385 [M+H]⁺; Rₜ=3.31 min (Method B).

Microanalysis for C₁₅H₁₇N₄O₂SCI:

Calc: C, 46.81%; H, 4.45%; N, 14.56%;

Found: C, 46.83%; H, 4.46%; N, 14.36%.

### Example 8

{4-Amino-2-[3-(4-methylpiperazin-1-yl)propylamino]thiazol-5-yl}-(5-chlorothiophen-2-yl)methanone

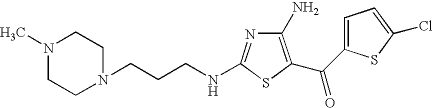

The starting material, 1-(3-isothiocyanatopropyl)₄-methylpiperazine, was first prepared, from the corresponding amine, using di-2-pyridylthionocarbonate, according to the method of Kim, S. and Yi, K. Y. (Tetrahedron Lett., 26 (13), 1985, pp. 1661-1664). The crude product obtained by precipitation from water, was dissolved in ethanol and treated with an excess of HCl gas in diethylether (ca. 2 M) to give the title compound as a hydrochloride salt in 13% yield.

M.p. 210-212° C.; HPLC-MS (ESI): m/z 401 [M+H]⁺; Rₜ=1.65 min (Method B).

### Example 9

{4-Amino-2-[3-(4-methylpiperazin-1-yl)propylamino]thiazol-5-yl}-(5-chlorothiophen-2-yl)methanone

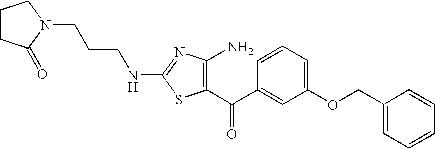

The reaction was performed according to Example 6, with the ring-opened intermediate first being isolated after addition of water to the reaction mixture. This was dissolved in hot ethanol and treated with 1.2 equivalents of triethlyamine. After 2 hours, reaction was cooled and filtered to furnish the title compound in 32% yield as a white solid.

M.p. 184° C.; ¹H NMR (300 MHz; DMSO-d₆): δ 1.73 (2H, ddd, CH₂), 1.92 (2H, ddd, CH₂), 2.20 (2H, t, CH₂), 3.23 (4H, m, 2×CH₂), 3.38 (2H, t, CH₂), 5.15 (2H, s, OCH₂), 7.08-7.48 (9H, m, Ar—H), 7.81-8.4 (2H, br d, NH₂), 8.58 (1H, br t, NH); HPLC-MS (ESI): m/z 451 [M+H]⁺; Rₜ=3.47 min (Method B).

### Example 10

{4-Amino-2-[3-(4-methylpiperazin-1-yl)propylamino]thiazol-5-yl}-(3-benzyloxyphenyl)methanone

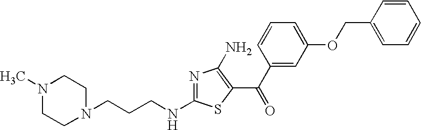

The reaction and work-up were performed as described in Example 8. The title compound was obtained as a hydrochloride salt.

¹H NMR (300 MHz; DMSO-d₆): δ 2.04 (2H, ddd, CH₂), 2.85 (3H, s, CH₃), 3.22 (2H, br, CH₂), 3.41 (6H, m, 3×CH₂), 3.70 (4H, m, CH₂), 5.17 (2H, s, OCH₂), 7.08-7.48 (9H, m, Ar—H), 7.81-8.35 (2H, br d, NH₂), 8.81 (1H, br t, NH), 11.88 (1H, br s, NH); M.p. 99-100° C.; HPLC-MS (ESI): m/z 466 [M+H]⁺; Rₜ=2.30 min (Method B).

### Example 11

[4-Amino-2-(3-aminopropylamino)thiazol-5-yl]cyclopropylmethanone

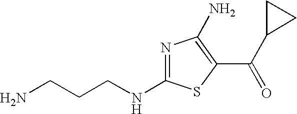

The product from Example 1, [3-(4-amino-5-cyclopropanecarbonylthiazol-2-yl-amino)propyl]carbamic acid tert-butyl ester (100 mg; 0.30 mmol), was dissolved in ethanol (5 ml) and treated with a three-fold excess of a solution of HCl gas in diethyl ether (ca. 2M). White solid was filtered off and washed with ether giving the title compound as the hydrochloride salt in 80% yield.

¹H NMR (300 MHz; DMSO-d₆): δ 0.75 (2H, m, CH₂), 0.82 (2H, m, CH₂), 1.64 (1H, m, CH), 1.88 (2H, ddd, CH₂), 2.84 (2H, dd, CH₂), 3.34 (2H, dd, CH₂), 5.20-6.0 (3H, br s, NH₃), 8.03 (2H, br s, NH₂), 8.75 (1H, t, NH); HPLC-MS (ESI): m/z 241 [M+H]⁺; Rₜ=0.43 and 2.68 (Method B).

### Example 12

[4-Amino-2-(2-aminoethylamino)thiazol-5-yl]cyclopropylmethanone

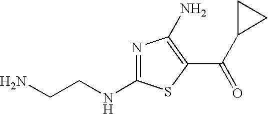

The title compound was obtained as a hydrochloride salt, starting from [2-(4-amino-5-cyclopropanecarbonylthiazol-2-ylamino)ethyl]carbamic acid tert-butyl ester and using the method described in Example 11.

¹H NMR (300 MHz; DMSO-d₆): δ 0.77 (2H, m, CH₂), 0.81 (2H, m, CH₂), 1.66 (1H, m, CH), 3.04 (2H, dd, CH₂), 3.49 (2H, dd, CH₂), 4.42 (3H, br s, NH₃), 8.13 (2H, br s, NH₂), 8.75 (1H, t, NH); HPLC-MS (ESI): m/z 227 [M+H]⁺; Rₜ=0.47 min (Method B).

### Example 13

[4-Amino-2-(3-aminopropylamino)thiazol-5-yl]-(5-chlorothiophen-2-yl)methanone

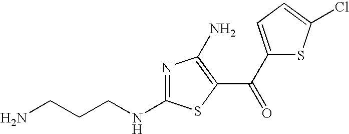

The product from Example 3, 3-[4-amino-5-(5-chlorothiophene-2-carbonyl)thiazol-2-ylamino]propylcarbamic acid tert-butyl ester (51 mg; 0.12 mmol), was dissolved in dichloromethane (1 ml) and treated with TFA (0.5 ml). After 2 hours at room temperature, solvents were evaporated to afford the title compound as a TFA salt.

¹H NMR (300 MHz; DMSO-d₆): δ 1.86 (2H, ddd, CH₂), 2.88 (2H, dd, CH₂), 3.40 (2H, br s, CH₂), 7.18 (1H, d, Ar—H), 7.33 (1H, d, Ar—H), 7.76 (3H, br s, NH₃), 8.1 and 8.45 (2H, br, 2×s, NH₂), 8.95 (1H, br t, NH); HPLC-MS (ESI): m/z 317 [M+H]⁺; Rₜ=1.82 min.

### Example 14

[4-Amino-2-(piperidin-4-ylamino)thiazol-5-yl]cyclopropylmethanone

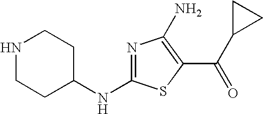

The title compound was obtained as a TFA salt, starting from 4-(4-amino-5-cyclopropanecarbonylthiazol-2-ylamino)piperidine-1-carboxylic acid tert-butyl ester, and using the method described in Example 13.

HPLC-MS (ESI): m/z 267 [M+H]⁺; Rₜ=0.65 min.

### Example 15

[4-Amino-2-(piperidin-4-ylamino)-thiazol-5-yl]-(3-benzyloxyphenyl)-methanone

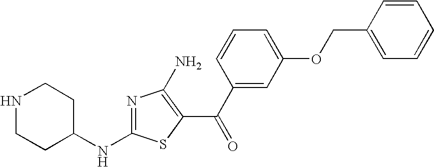

The title compound was obtained as a TFA salt, starting from 4-[4-amino-5-(3-benzyloxybenzoyl)thiazol-2-ylamino]piperidine-1-carboxylic acid tert-butyl ester, and using the method described in Example 13.

HPLC-MS (ESI): m/z 409 [M+H]⁺; Rₜ=2.33 min.

### Example 16

[4-Amino-2-(3-aminopropylamino)thiazol-5-yl]-(3-benzyloxyphenyl)methanone

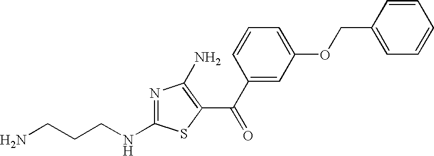

The title compound was obtained as a TFA salt, starting from {3-[4-amino-5-(3-benzyloxybenzoyl)thiazol-2-ylamino]propyl}carbamic acid tert-butyl ester, and using the method described in Example 13.

¹H NMR (300 MHz; DMSO-d₆): δ 1.85 (2H, ddd, CH₂), 2.83 (2H, dd, CH₂), 3.33 (2H, br, CH₂), 5.18 (2H, s, OCH₂), 6.58 (3H, br s, NH₃), 7.11-7.49 (9H, m, Ar—H), 7.95 (2H, br s, NH₂), 8.80 (1H, br t, NH); HPLC-MS (ESI): m/z 383 [M+H]⁺; Rₜ=2.30 min.

The following compounds are also within the scope of the present invention:

[4-Amino-2-(1-methyl-1,2,5,6-tetrahydro-pyridin-3-ylamino)thiazol-5-yl]phenyl-methanone

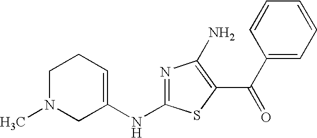

[4-Amino-2-(1-benzylpiperidin-4-yl-amino)thiazol-5-yl]-(5-chlorothiophen-2-yl)methanone

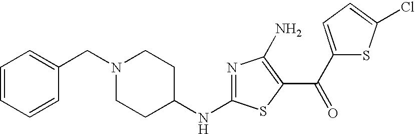

[4-Amino-2-(1-benzylpiperidin-4-ylamino)-thiazol-5-yl]-(3-benzyloxyphenyl)methanone

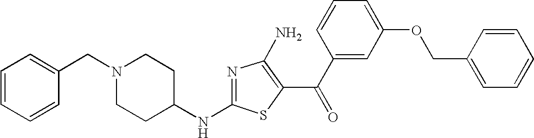

{3-[4-Amino-5-(3,4-difluorobenzyl)-thiazol-2-ylamino]propyl}carbamic acid tert-butyl ester

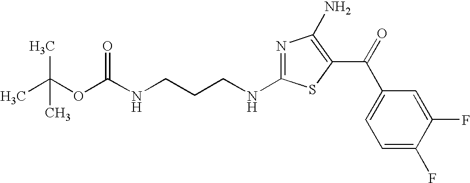

(3-{4-Amino-5-[3-(2-chlorothiazol-4-yl-methoxy)benzoyl]thiazol-2-ylamino}propyl)-carbamic acid
tert-butyl ester

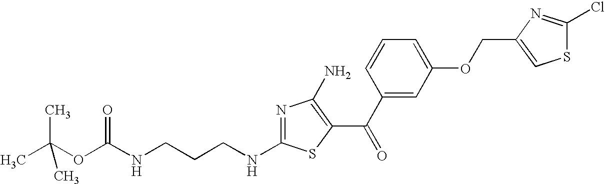

1-{3-[4-Amino-5-(3-hydroxybenzoyl)-thiazol-2-ylamino]propyl}pyrrolidin-2-one

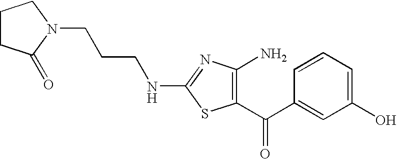

N-{3-[4-Amino-5-(3-benzyloxybenzoyl)thiazol-2-ylamino]propyl}acetamide

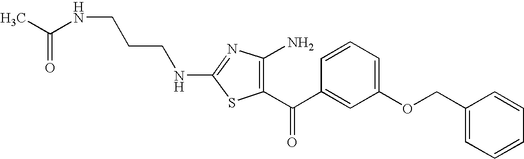

1-{3-[4-Amino-5-(5-chlorothiophene-2-carbonyl)thiazol-2-ylamino]propyl}pyrrolidine-2,5-dione

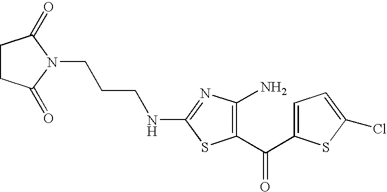

Pharmacological Methods

Assay (I)

Inhibition of GSK-3 by a test compound was evaluated using human GSK-3β and a glycogen synthase derived substrate with the following amino acid sequence:

YRRAAVPPSPSLSRHSSPHQS(PO₄)EDEEE-NH₂

In brief, GSK-3β was incubated with 35 μM substrate and varying concentrations of test compound in a buffer containing 0.1 mM ³³P-labeled ATP, 10 mM magnesium acetate, 8 mM MOPS pH 7.0, 0.2 mM EDTA, 0.1% dithiothreitol and 0.03% Triton-X100 for 60 min at room temperature. The reaction was performed using 96-well plates. The reaction was terminated by adding 13 μl 2% phosphoric acid to each well, and 10 μl was spotted onto P30 paper which was washed 4 times in 0.5% phosphoric acid to remove non-incorporated ³³P-labeled ATP. After drying the radioactivity was counted in a Wallac. Dose-response profiles were generated, and the IC₅₀ value for inhibition of GSK-3 by the test compound was calculated using a four-parameter logistic function.

The following compounds inhibited GSK-3 with an IC₅₀ value lower than 1 μM:

Examples 3, 5, 6, 7, 9 and 10.

From the foregoing, it will be appreciated that, although specific embodiments of the invention have been described herein for the purposes of illustration, various modifications may be made without deviating from the spirit and scope of the invention as defined by the appending claims.

1. A compound of formula (I): 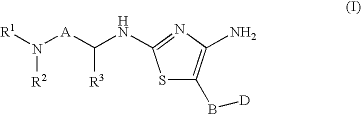
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆-alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₄-alkynyl,
which may optionally be substituted with one or two substituents selected from hydroxy, halogen, cyano, nitro, —NR²⁴R²⁵—C(═O)NR²⁴R²⁵—OC(═O)NR²⁴R²⁵—OCH₂C(═O)NR²⁴R²⁵, C₁-alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷—C(═O)NR²⁶R²⁷, —OC(═O)NR²⁶R²⁷—OCH₂C(═O)NR²⁶R²⁷, C₁₋₆-alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the
nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆-alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³OR³, —OC(═O)NR³OR³, —OCH₂C(═O)NR³OR³, C₁₋₄-alkyl, C₂₋₄-alkenyl, C₂₋₄-alkynyl, C₁₋₆alkoxy, —C(═O)OR³, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
with the proviso that the compound must not be 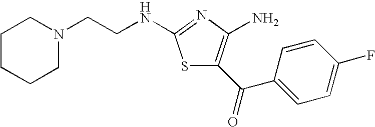
as well as any optical or geometric isomer or tautomeric form thereof including mixtures of these or a pharmaceutically acceptable salt thereof.

2. A compound according to claim 1 wherein R² and R³ are both hydrogen, and R¹ is —C(═O)OR¹⁹.

3. A compound according to claim 2 wherein R¹⁹ is C₁₋₆-alkyl.

4. A compound according to claim 1 wherein R³ is hydrogen, and R¹ and R², together with the nitrogen atom to which they are attached, form a ring

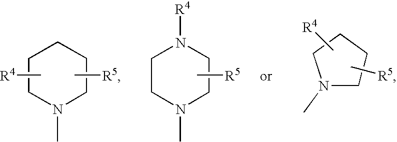

5. A compound according to claim 4 wherein R⁴ and R⁵ are independently selected from hydrogen, C₁₋₆-alkyl, phenyl-C₁₋₆-alkyl and oxo.

6. A compound according to claim 5 wherein R⁴ is hydrogen or Cl-alkyl, and R⁵ is hydrogen or oxo.

7. A compound according to claim 4 wherein R³ is hydrogen, and R¹ and R², together with the nitrogen atom to which they are attached, form a ring

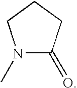

8. A compound according to claim 1 wherein R² and R³, together with A and the nitrogen atom and the carbon atom, respectively, to which they are attached, form a ring

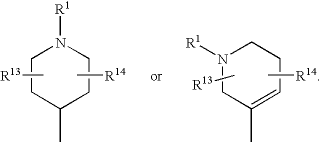

9. A compound according to claim 8 wherein R² and R³, together with A and the nitrogen atom and the carbon atom, respectively, to which they are attached, form a ring

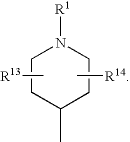

10. A compound according to claim 8 or 9 wherein R¹ is hydrogen, C₁₋₆-alkyl, phenyl-C₁₋₆-alkyl or —C(═O)OR¹⁰, wherein R¹⁰ is as defined in claim 1, and R¹³ and R¹⁴ are independently hydrogen, C₁₋₆-alkyl, phenyl-C₁₋₆-alkyl or oxo.

11. A compound according to claim 10 wherein R¹ is hydrogen or —C(═O)O—C₁₋₆-alkyl, and R¹³ and R¹⁴ are hydrogen.

12. A compound according to claim 1 wherein R¹, R² and R³ are hydrogen.

13. A compound according to claim 1 wherein A is C₁₋₆-alkylene.

14. A compound according to claim 13 wherein A is methylene or ethylene.

15. A compound according to claim 14 wherein A is ethylene.

16. A compound according to claim 1 wherein B is —C(═O)—.

17. A compound according to claim 1 wherein D is C₃₋₈-cycloalkyl, heteroaryl or aryl, which may optionally be substituted.

18. A compound according to claim 1 wherein D is C₃₋₈-cycloalkyl, heteroaryl or aryl, which may optionally be substituted, but not in the positions adjacent to the point of attachment of D to B.

19. A compound according to claim 17 or 18 wherein D is cyclopropyl, thienyl or phenyl, which may optionally be substituted.

20. A compound according to claim 17 wherein D is cyclopropyl.

21. A compound according to claim 17 or 18 wherein D is thienyl, which is substituted with halogen.

22. A compound according to claim 17 or 18 wherein D is phenyl, which is optionally substituted with
hydroxy, halogen,
heteroaryl-C₁₋₆-alkoxy, aryl-C₁₋₆-alkoxy, wherein the ring moieties are optionally substituted.

23. A compound according to claim 22 wherein D is phenyl which is optionally substituted with halogen or benzyloxy, wherein the ring moiety of benzyloxy is optionally substituted.

24. A compound according to claim 23 wherein D is phenyl, which is substituted with benzyloxy.

25. A pharmaceutical composition comprising at least one compound according to claim 1 together with one or more pharmaceutically acceptable carriers or excipients.

26. A pharmaceutical composition according to claim 25 in unit dosage form, comprising from about 0.05 mg to about 1000 mg of the compound according to claim 1.

27. A method for treating diseases, disorders, syndromes and conditions wherein an inhibition of glycogen synthase kinase-3 (GSK-3) is beneficial, said method comprising administering to a subject in need thereof an effective amount of a compound of formula (I′): 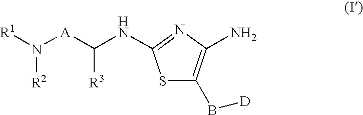
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆-alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₄-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆-alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆-alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵, —OC(═O)NR²⁴R²⁵—OCH₂C(═O)NR²⁴R²⁵, C₁₋₆alkoxy, —C(═O)OR²⁴, —C(═O)R²⁴, —NHC(═O)R²⁴, —CHF₂—CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷—C(═O)NR²⁶R²⁷—OC(═O)NR²⁶R²⁷—OCH₂C(═O)NR²⁶R²⁷, C₁₋₆alkoxy, —C(═O)OR²⁶, —C(═O)R²⁶, —NHC(═O)R²⁶, —CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆-alkoxy, —C(═O)OR², —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cyclo-alkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₆-heterocyclyl, —S-aryl, —S—C₃₋₈-cycloalkyl, —S-heteroaryl, —S—C₃₋₆-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl,
C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

28. The method according to claim 27, wherein the effective amount of the compound is in the range of from about 0.05 mg to about 2000 mg per day.

29. A pharmaceutical composition according to claim 25 in unit dosage form; comprising from about 0.1 mg to about 500 mg of the compound according to claim 1.

30. A pharmaceutical composition according to claim 25 in unit dosage form, comprising from about 0.5 mg to about 200 mg of the compound according to claim 1.

31. The method according to claim 27, wherein the effective amount of the compound is in the range of from about 0.1 mg to about 1000 mg per day.

32. The method according to claim 27, wherein the effective amount of the compound is in the range of from about 0.5 mg to about 500 mg per day.

33. A method for treating diseases, disorders, syndromes and conditions related to glycogen synthase kinase-3 (GSK-3), said method comprising administering to a subject in need thereof an effective amount of a compound of formula (I′): 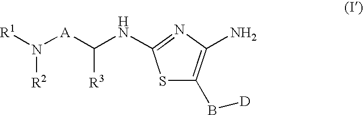
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆-alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₄-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵, —OC(═O)NR²⁴R²⁵—OCH₂C(═O)NR²⁴R², C₁₋₆alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —N H-aryl, —N H-heteroaryl,
wherein the ring moieties may optionally be substituted with one to three substituents selected from
hydroxy, halogen, cyano, nitro, —NR²⁶R²⁷, —C(═O)NR²⁶R²⁷, —OC(═O)NR²⁶R²⁷—OCH₂C(═O)NR²⁶R²⁷, C₁₋₆alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁵ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₄-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cyclo-alkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰—S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

34. A method for treating diseases, disorders, syndromes and conditions wherein growth factor induced inhibition of glycogen synthase kinase-3 (GSK-3) is insufficient, said method comprising administering to a subject in need thereof an effective amount of a compound of formula (I′): 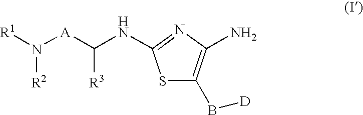
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆-alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆alkenyl, C₂₋₆-alkynyl, C₁₋₆alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰OR²¹, —OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵, —OC(═O)NR²⁴R²⁵—OCH₂C(═O)NR²⁴R²⁵, C₁₋₆alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷, —C(═O)NR²⁶R²⁷, —OC(═O)NR²⁶R²⁷—OCH₂C(═O)NR²⁶R²⁷, C₁₋₆alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁵ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

35. A method for treating diseases, disorders, syndromes and conditions wherein glycogen metabolism exhibits abnormalities, said method comprising administering to a subject in need thereof an effective amount of a compound of formula (I′): 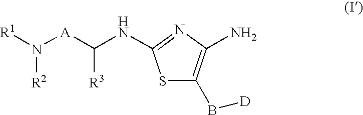
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶—OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆-alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹—OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³—C(═O)NR²²R²³—OC(═O)NR²²R²³, —OCH₂C(═O)NR²²R²³, C₁₋₆-alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵—C(═O)NR²⁴R²⁵—OC(═O)NR²⁴R²⁵—OCH₂C(═O)NR²⁴R²⁵, C₁₋₆-alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷, —C(═O)NR²⁶R²⁷, —OC(═O)NR²⁶R²⁷—OCH₂C(═O)NR²⁶R²⁷, C₁₋₆-alkoxy, —C(═O)OR²⁶, —C(═O)R²⁶, —NHC(═O)R²⁶, —CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆-alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰—C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

36. A method for treating diseases, disorders, syndromes and conditions wherein glycogen synthase is insufficiently activated, said method comprising administering to a subject in need thereof an effective amount of a compound of formula (I′): 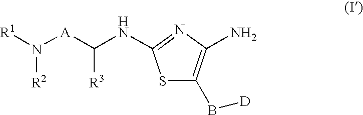
wherein
A is a valence bond or C₁-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆-alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆alkenyl, C₂₋₆-alkynyl, C₁₋₆alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵—OC(═O)NR²⁴R²⁵—OCH₂C(═O)NR²⁴R²⁵, C₁₋₆-alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-Cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷—C(═O)NR²⁶R²⁷—OC(═O)NR²⁶R²⁷—OCH₂C(═O)NR²⁶R²⁷, C₁₋₆-alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰OR³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

37. A method for treating diseases, disorders, syndromes and conditions involving elevated blood glucose, said method comprising administering to a subject in need thereof an effective amount of a compound of formula (I′): 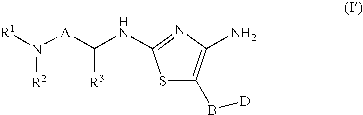
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-G₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵—C(═O)NR²⁴R²⁵—OC(═O)NR²⁴R²⁵—OCH₂C(═O)NR²⁴R²⁵, C₁₋₆alkoxy, —C(═O)OR²⁴, —C(═O)R²⁴, —N HC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷, —C(═O)NR²⁶R²⁷, —OC(═O)NR²⁶R²⁷—OCH₂C(═O)NR²⁶R²⁷, C₁₋₆-alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆-alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

38. A method for treating hyperglycemia, said method comprising administering to a subject in need thereof an effective amount of a compound of formula (I′): 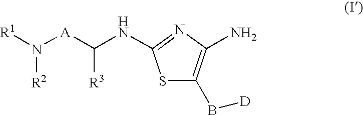
wherein
A is a valence bond or C₁-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R⁵, —S(═O)₂R⁵, —S(═O)₂NH₂,
wherein R¹⁵ and R¹⁶ which may be the same or different independently are selected from hydrogen and C₁₋₆-alkyl, or R¹⁵ and R⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³, —OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵, —OC(═O)NR²⁴R²⁵, —OCH₂C(═O)NR²⁴R²⁵, C₁₋₆alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷—C(═O)NR²⁶R²⁷—OC(═O)NR²⁶R²⁷—OCH₂C(═O)NR²⁶R²⁷C₁₋₆alkoxy, —C(═O)OR²⁶, —C(═O)R²⁶, —NHC(═O)R²⁶, —CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆-alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-Cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

39. A method for treating impaired glucose tolerance, said method comprising administering to a subject in need thereof an effective amount of a compound of formula (I′): 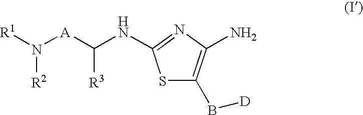
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆-alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆-alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₃₋₈-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵, —OC(═O)NR²⁴R²⁵—OCH₂C(═O)NR²⁴R²⁵, C₁₋₆-alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷, —C(═O)NR²⁶R²⁷, —OC(═O)NR²⁶R²⁷—OCH₂C(═O)NR²⁶R²⁷, C₁₋₆alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R², —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹, —OCH₂C(═O)NR²⁸R²⁹, C₁₋₆-alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

40. A method for treating type 2 diabetes, said method comprising administering to a subject in need thereof an effective amount of a compound of formula (I′): 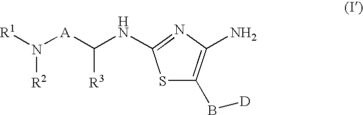
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₁₋₆-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₄-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹—OCH₂C(═O)NR²⁰R²¹, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵—OC(═O)NR²⁴R²⁵—OCH₂C(═O)NR²⁴R²⁵, C₁₋₆-alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷—C(═O)NR²⁶R²⁷, —OC(═O)NR²⁶R²⁷—OCH₂C(═O)NR²⁶R²⁷, C₁₋₆alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹, —OCH₂C(═O)NR²⁸R²⁹, C₁₋₆-alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

41. A method for treating type 1 diabetes, said method comprising administering to a subject in need thereof an effective amount of a compound of formula (I′): 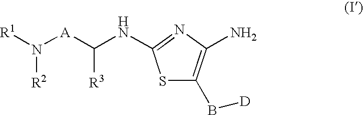
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆₋-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹²—OC(═O)NR¹¹R¹²—OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵RR¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵, —OC(═O)NR²⁴R²⁵—OCH₂C(═O)NR²⁴R²⁵, C₁₋₆-alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷—C(═O)NR²⁶R²⁷—OC(═O)NR²⁶R²⁷—OCH₂C(═O)NR²⁶R²⁷, C₁₋₆-alkoxy, —C(═O)OR²⁶, —C(═O)R²⁶, —NHC(═O)R²⁶, —CHF₂, —CF₃, —OCF₃, —OCHF₂, —OCH₂CF₃, —OCF₂CHF₂, —SCF₃, —SR²⁶, —S(═O)R², —S(═O)₂R²⁶, —S(═O)₂NH₂,
wherein R²⁵ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹, —OCH₂C(═O)NR²⁸R²⁹, C₁₋₆-alkoxy, —C(═O)OR²⁸, —C(═O)R², —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

42. A method for treating obesity, said method comprising administering to a subject in need thereof an effective amount of a compound of formula (I′): 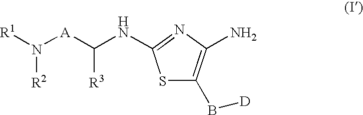
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R¹⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-Cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-Cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from
hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆-alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆alkoxy, —C(═O)OR²⁰, —C(═O)R²⁰, —NHC(═O)R², —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵—OC(═O)NR²⁴R²⁵—OCH₂C(═O)NR²⁴R²⁵, C₁₋₆-alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-Cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷—C(═O)NR²⁶R²⁷—OC(═O)NR²⁶R²⁷—OCH₂C(═O)NR²⁶R²⁷, C₁₋₆-alkoxy, —C(═O)OR²⁶, —C(═O)R²⁶, —NHC(═O)R²⁶, —CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₆-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-Cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

43. A method for treating Alzheimer's disease, said method comprising administering to a subject in need thereof an effective amount of a compound of formula (I′): 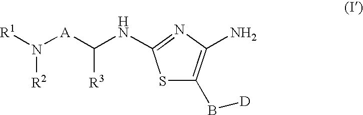
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆-alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R¹², —OC(═O)NR¹¹R¹², —OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁵R⁶, —C(═O)NR⁵R¹⁶, —OC(═O)NR⁵R⁶, —OCH₂C(═O)NR⁵R⁶, C₁₋₆alkoxy, —C(═O)OR⁵, —C(═O)R¹⁵, —NHC(═O)R⁵, —CHF₂, —CF₃, —OCF₃, —OCHF₂, —OCH₂CF₃, —OCF₂CHF₂, —SCF₃, —SR¹⁵, —S(═O)R⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³—N(R²²)OR²³—C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵—C(═O)NR²⁴R²⁵—OC(═O)NR²⁴R²⁵—OCH₂C(═O)NR²⁴R²⁵, C₁₋₆alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷, —C(═O)NR²⁶R²⁷, —OC(═O)NR²⁶R²⁷—OCH₂C(═O)NR²⁶R²⁷, C₁₋₆alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆alkoxy, —C(═O)OR², —C(═O)R², —NHC(═O)R²⁸, —CHF₂, —CF₃, —OCF₃, —OCHF₂, —OCH₂CF₃, —OCF₂CHF₂, —SCF₃, —SR²³, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²³ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³³, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

44. A method for treating bipolar disorder, said method comprising administering to a subject in need thereof an effective amount of a compound of formula (I′): 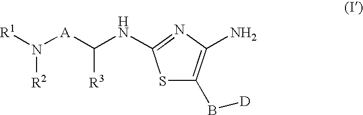
wherein
A is a valence bond or C₁₋₆-alkylene,
(i) R¹ and R², together with the nitrogen atom to which they are attached, form a 5 to 7 membered non-aromatic ring, which ring may optionally contain a double bond, and which ring may optionally contain a further nitrogen atom, and to which ring is attached two groups R⁴ and R⁵ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR⁶R⁷, —C(═O)NR⁶R⁷, —OC(═O)NR⁶R⁷, —OCH₂C(═O)NR⁶R⁷, C₁₋₆alkoxy, —C(═O)OR⁶, —C(═O)R⁶, —NHC(═O)R⁶, —CHF₂, —CF₃, —OCF₃, —OCHF₂, —OCH₂CF₃, —OCF₂CHF₂, —SCF₃, —SR⁶, —S(═O)R⁶, —S(═O)₂R⁶, —S(═O)₂NH₂,
wherein R⁶ and R⁷ which may be the same or different independently are selected from hydrogen and C₁₋₆-alkyl, or R⁶ and R⁷, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR⁸R⁹, —C(═O)NR⁸R⁹, —OC(═O)NR⁸R⁹, —OCH₂C(═O)NR⁸R⁹, C₁₋₆alkoxy, —C(═O)OR⁸, —C(═O)R⁸, —NHC(═O)R⁸, —CHF₂, —CF₃, —OCF₃, —OCHF₂, —OCH₂CF₃, —OCF₂CHF₂, —SCF₃, —SR⁸, —S(═O)R⁸, —S(═O)₂R⁸, —S(═O)₂NH₂,
wherein R⁸ and R⁹ which may be the same or different independently are selected from hydrogen and C₁₋₆-alkyl, or R⁸ and R⁹, together with the nitrogen atom to which they are attached, form a 3 to 8 membered cyclic ring optionally containing one or two further heteroatoms selected from oxygen, sulphur and nitrogen,
and R³ is hydrogen,
(ii) or R¹ is hydrogen, —C(═O)OR¹⁰, —C(═O)R¹⁰, C₁₋₆-alkyl, aryl-C₁₋₆-alkyl, C₃₋₈-cycloalkyl-C₁₋₆-alkyl, heteroaryl-C₁₋₆-alkyl or C₃₋₈-heterocyclyl-C₁₋₆-alkyl,
wherein R¹⁰ is C₁₋₆-alkyl, C₂₋₆-alkenyl or C₂₋₆-alkynyl, which may optionally be substituted with one or two substituents independently selected from
hydroxy, halogen, cyano, nitro, —NR¹¹R¹², —C(═O)NR¹¹R², —OC(═O)NR¹¹R¹², —OCH₂C(═O)NR¹¹R¹², C₁₋₆-alkyl, C₂₋₆-alkenyl, C₂₋₆-alkynyl, C₁₋₆-alkoxy, —C(═O)OR¹¹, —C(═O)R¹¹, —NHC(═O)R¹¹, —CHF₂, —CF₃, —OCF₃, —OCHF₂, —OCH₂CF₃, —OCF₂CHF₂, —SCF₃, —SR¹¹, —S(═O)R¹¹, —S(═O)₂R¹¹, —S(═O)₂NH₂,
wherein R¹¹ and R¹² which may be the same or different independently are selected from hydrogen and C₁₋₆-alkyl, or R¹¹ and R¹², together with the nitrogen atom to which they are attached, form a 3 to 8 membered cyclic ring optionally containing one or two further heteroatoms selected from oxygen, sulphur and nitrogen,
and R² and R³ are connected to form, together with A and the nitrogen atom and carbon atom, respectively, to which they are attached, a 5 to 7 membered non-aromatic ring to which ring is attached two groups R¹³ and R¹⁴ which are independently selected from
hydrogen,
oxo,
C₁₋₆-alkyl, C₂₋₆-alkenyl, C₂₋₆-alkynyl,
which may optionally be substituted with one or two substituents independently selected from hydroxy, halogen, cyano, nitro, —NR¹⁵R¹⁶, —C(═O)NR¹⁵R¹⁶, —OC(═O)NR¹⁵R⁶, —OCH₂C(═O)NR¹⁵R¹⁶, C₁₋₆alkoxy, —C(═O)OR¹⁵, —C(═O)R¹⁵, —NHC(═O)R¹⁵, —CHF₂, —CF₃, —OCF₃, —OCHF₂, —OCH₂CF₃, —OCF₂CHF₂, —SCF₃, —SR¹⁵, —S(═O)R¹⁵, —S(═O)₂R¹⁵, —S(═O)₂NH₂,
wherein R¹⁵ and R¹⁶ which may be the same or different independently are selected from hydrogen and C₁₋₆-alkyl, or R¹⁵ and R¹⁶, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents independently selected from hydroxy, halogen, cyano, nitro, —NR¹⁷R¹⁸, —C(═O)NR¹⁷R¹⁸, —OC(═O)NR¹⁷R¹⁸, —OCH₂C(═O)NR¹⁷R¹⁸, C₁₋₆-alkyl, C₂₋₆-alkenyl, C₂₋₆-alkynyl, C₁₋₆alkoxy, —C(═O)OR¹⁷, —C(═O)R¹⁷, —NHC(═O)R¹⁷, —CHF₂, —CF₃, —OCF₃, —OCHF₂, —OCH₂CF₃, —OCF₂CHF₂, —SCF₃, —SR¹⁷, —S(═O)R¹⁷, —S(═O)₂R¹⁷, —S(═O)₂NH₂,
wherein R¹⁷ and R¹⁸ which may be the same or different independently are selected from hydrogen and C₁₋₆-alkyl, or R¹⁷ and R¹⁸, together with the nitrogen atom to which they are attached, form a 3 to 8 membered cyclic ring optionally containing one or two further heteroatoms selected from oxygen, sulphur and nitrogen,
(iii) or R¹ and R² which may be the same or different independently are selected from hydrogen, —C(═O)OR¹⁹—C(═O)R¹⁹ and C₁₋₆-alkyl,
wherein R¹⁹ is C₁₋₆-alkyl, C₂₋₆-alkenyl or C₂₋₆-alkynyl, which may optionally be substituted with one or two substituents independently selected from
hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl, or R²⁰ and R²¹, together with the nitrogen atom to which they are attached, form a 3 to 8 membered cyclic ring optionally containing one or two further heteroatoms selected from oxygen, sulphur and nitrogen,
and R³ is hydrogen,
B is a valence bond, —C(═O)—, —S(═O)— or —S(═O)₂—,
D is
hydroxy, halogen, cyano, nitro, —NR²²R²³, —N(R²²)OR²³, —C(═O)NR²²R²³—OC(═O)NR²²R²³, —OCH₂C(═O)NR²²R²³, C₁₋₆alkoxy, —C(═O)OR²², —C(═O)R²², —NHC(═O)R²², —CHF₂, —CF₃, —OCF₃, —OCHF₂, —OCH₂CF₃, —OCF₂CHF₂, —SCF₃, —SR²², —S(═O)R²², —S(═O)₂R²², —S(═O)₂NH₂,
wherein R²² and R²³ which may be the same or different independently are selected from hydrogen and C₁₋₆-alkyl, or R²² and R²³, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁴R²⁵, —C(═O)NR²⁴R²⁵—OC(═O)NR²⁴R²⁵—OCH₂C(═O)NR²⁴R²⁵, C₁₋₆alkoxy, —C(═O)OR²⁴, —C(═O)R²⁴, —NHC(═O)R²⁴, —CHF₂, —CF₃, —OCF₃, —OCHF₂, —OCH₂CF₃, —OCF₂CHF₂, —SCF₃, —SR²⁴, —S(═O)R²⁴, —S(═O)₂R²⁴, —S(═O)₂NH₂,
wherein R²⁴ and R²⁵ which may be the same or different independently are selected from hydrogen and C₁₋₆-alkyl, or R²⁴ and R²⁵, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
wherein the ring moieties may optionally be substituted with one to three substituents selected from
hydroxy, halogen, cyano, nitro, —NR²⁶R²⁷—C(═O)NR²⁶R²⁷—OC(═O)NR²⁶R²⁷—OCH₂C(═O)NR²⁶R²⁷, C₁₋₆-alkoxy, —C(═O)OR²⁶, —C(═O)R²⁶, —NHC(═O)R²⁶—CHF₂, —CF₃, —OCF₃, —OCHF₂, —OCH₂CF₃, —OCF₂CHF₂, —SCF₃, —SR²⁶, —S(═O)R²⁶, —S(═O)₂R²⁶, —S(═O)₂NH₂,
wherein R²⁶ and R²⁷ which may be the same or different independently are selected from hydrogen and C₁₋₆-alkyl, or R²⁶ and R²⁷, together with the nitrogen atom to which they are attached, form a 3 to 8 membered cyclic ring optionally containing one or two further heteroatoms selected from oxygen, sulphur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR²⁸R²⁹, —C(═O)NR²⁸R²⁹, —OC(═O)NR²⁸R²⁹—OCH₂C(═O)NR²⁸R²⁹, C₁₋₆-alkoxy, —C(═O)OR²⁸, —C(═O)R²⁸, —NHC(═O)R²⁸, —CHF₂, —CF₃, —OCF₃, —OCHF₂, —OCH₂CF₃, —OCF₂CHF₂, —SCF₃, —SR²⁸, —S(═O)R²⁸, —S(═O)₂R²⁸, —S(═O)₂NH₂,
wherein R²⁸ and R²⁹ which may be the same or different independently are selected from hydrogen and C₁₋₆-alkyl, or R²⁸ and R²⁹, together with the nitrogen atom to which they are attached, form a 3 to 8 membered cyclic ring optionally containing one or two further heteroatoms selected from oxygen, sulphur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, —C(═O)-aryl, —C(═O)—C₃₋₈-cycloalkyl, —C(═O)-heteroaroyl, —C(═O)—C₃₋₈-heterocyclyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
wherein the ring moieties may optionally be substituted with one to three substituents selected from hydroxy, halogen, cyano, nitro, —NR³⁰R³¹, —C(═O)NR³⁰R³¹, —OC(═O)NR³⁰R³¹, —OCH₂C(═O)NR³⁰R³¹, C₁₋₆-alkyl, C₂₋₆-alkenyl, C₂₋₆-alkynyl, C₁₋₆alkoxy, —C(═O)OR³⁰, —C(═O)R³⁰, —NHC(═O)R³⁰, —CHF₂, —CF₃, —OCF₃, —OCHF₂, —OCH₂CF₃, —OCF₂CHF₂, —SCF₃, —SR³⁰, —S(═O)R³⁰, —S(═O)₂R³⁰, —S(═O)₂NH₂,
wherein R³⁰ and R³¹ which may be the same or different independently are selected from hydrogen and C₁₋₆-alkyl, or R³⁰ and R³¹, together with the nitrogen atom to which they are attached, form a 3 to 8 membered cyclic ring optionally containing one or two further heteroatoms selected from oxygen, sulphur and nitrogen,
as well as any optical or geometric isomer or tautomeric form thereof including mixtures of these or a pharmaceutically acceptable salt thereof.

45. The method according to claims 27 and 33-42, said method comprising further administering to said subject one or more agents selected from the group consisting of antidiabetic agents, antihyperlipidemic compounds, antiobesity compounds and antihypertensive compounds.

46. The method according to claim 43, said method further comprising further administering to said subject one or more agents for treating Alzheimer's disease.

47. The method according to claim 44, said method further comprising further administering to said subject one or more agents for treating bipolar disorder.
